(19) United States
(12) Patent Application Publication
(10) Pub. No.: US 2022/0211404 A1
(43) Pub. Date: Jul. 7, 2022

(54) WET JET DEBRIDEMENT AND WOUND BED PREPARATION

(71) Applicant: SMITH & NEPHEW ASIA PACIFIC PTE. LIMITED, Singapore (SG)

(72) Inventors: Wee Chin Tay, Singapore (SG); Xiaodong Xu, Singapore (SG); Li Lan, Singapore (SG); Theng Theng Tan, Singapore (SG); Chee Seng Poon, Singapore (SG)

(21) Appl. No.: 17/595,679

(22) PCT Filed: Jun. 10, 2020
(86) PCT No.: PCT/IB2020/055443

§ 371 Date: Nov. 22, 2021

(30) Foreign Application Priority Data
Jun. 10, 2019 (GB) 1908251.0

(51) Int. Cl. A61B 17/3203

(52) U.S. Cl. CPC A61B 17/3203

(57) ABSTRACT

A water jet debridement and wound bed preparation system includes a handle (22b) with integrated, molded components, for example, a distal tip (26) that receives liquid from a jet tube (28) is integral with a handle housing to which the jet tube is mounted. In another embodiment, in which the handle does not include a distal tip, liquid flows from a jet tube having a straight distal end region. In another embodiment, the system includes a piston pump with integrated, molded components. In another embodiment, the system includes a dual-spike saline bag assembly.

This application claims priority to GB1908251.0, filed Jun. 10, 2019, hereby incorporated by reference in its entirety.

### BACKGROUND OF THE INVENTION

The Smith & Nephew Versajet™ Hydrosurgery system use a high pressure saline water jet for debridement and wound bed preparation. Components of the Versajet™ Hydrosurgery system are described in U.S. Pat. Nos. 9,597,107 and 9,341,184, hereby incorporated by reference in their entirety. Referring to prior art FIGS. 1A-1C, saline water flows from a high pressure jet tube 11 at the top of the handle, turns 180° through the end 12 of the jet tube 11, and is ejected from the jet tube 11 to an evacuation tube 7 at the bottom of the handle. The jet tube 11 is welded to a distal tip 5 that defines a treatment window 14.

An orifice member (nozzle) 6 in the jet tube 11 is a key component in determining the water pressure and debridement effect. In the Versajet™ Hydrosurgery system, the orifice member 6 is assembled to the jet tube 11 by a crimping process. The orifice member 6 is placed on a flared end of the jet tube 11, and the end of the jet tube 11 is crimped by being bent inward to secure the orifice member 6 in the jet tube 11.

The orifice member is in the form of a ring configured to form a liquid jet and defines a liquid flow passage having a diameter that continuously decreases from a first terminal end to a location proximate to a second terminal end. The evacuation tube has an opening positioned opposite the orifice member and is configured to receive at least a portion of the liquid jet emitted from the orifice member and to convey a flow of liquid away from the opening. The pressure jet tube is configured and positioned to convey the flow of liquid to the orifice member. The pressure jet tube is mounted on an exterior surface of the housing and includes a holder located at a distal opening of the pressure jet tube. The holder forms a recessed well in the distal tip of the pressure jet tube and is configured to retain and position the orifice member completely inside the holder such that the orifice member is co-axial with a distal end of the pressure jet tube and the flow of liquid. The distal end of the opening of the pressure jet tube extends beyond a distal end of the evacuation tube such that, in operation, the flow of liquid from the orifice member is directed toward the handle.

A nozzle assembly is fabricated by affixing the orifice member in the shape of torus having a flat surface and a curved opposite surface to or within the holder. The nozzle assembly is able to withstand an internal liquid pressure of at least about 1,000 psig without failure.

As illustrated in prior art FIG. 2, the Versajet™ Hydrosurgery system includes a piston pump implemented into the single use hand piece. The pump includes a pump housing configured for removable receipt by a drive console. The pump housing including an insert section, a coupling section and a handle. The insert section, coupling section and handle are linearly arranged with the coupling section between the insert section and the handle. The insert section is configured to removably receive a push rod of the drive console. The coupling section includes an external oval flange. A valve assembly located in the pump housing includes an inlet passage, an outlet passage, an inlet ball valve, and an outlet ball valve. The inlet and outlet passages are located side-by-side in the pump housing and are in fluid communication with a chamber defined in the insert section. The chamber has a sloped wall is axially aligned with the inlet passage and the outlet passage. A piston is slidably received within the chamber and includes flexible members arranged within the chamber to be acted upon by the sloped wall to engage the push rod. The only external force required to couple the piston to the push rod is an axial force on the piston in a first direction, and the only external force required to de-couple the piston from the push rod is an axial force on the piston in a second direction opposite the first direction.

As illustrated in prior art FIG. 3, the Versajet™ Hydrosurgery system uses a single spike to connect with a saline bag.

### SUMMARY

The handle of the Versajet™ Hydrosurgery system hand piece includes multiple parts requiring a complex assembly/weldment process. In particular, the distal tip 5 (FIGS. 1A-1C) of the Versajet™ Hydrosurgery system has a complex geometry formed by metal injection molding and requires laser welding to the thin jet tube 11.

The current design reduces the number of parts and sub-assemblies, for example, several parts are integrated into one piece to avoid the assembly of the parts and maintain alignment of the parts.

A water jet hand piece for treating tissue includes a handle having an upper housing and a lower housing, a jet tube mounted to the upper housing, and a distal tip that receives liquid from the jet tube and defines a treatment window for treating tissue with a liquid jet. The distal tip is integral with the upper housing.

The top to bottom water flow in the Versajet™ Hydrosurgery system requires bending of the tip of the jet tube 11 as well as welding of the jet tube 11 to the distal tip 5.

A particular embodiment of the current design switches the position of the jet tube and the evacuation tube, allowing a jet tube with a straight distal end region and the elimination of the distal tip.

A water jet hand piece for treating tissue includes a handle including an upper housing and a lower housing. The lower housing defines a treatment window. The hand piece includes a jet tube mounted to the lower housing. The jet tube has a straight distal end region. The treatment window is configured for treating tissue with a liquid jet delivered to the treatment window via the jet tube. The hand piece includes an evacuation tube mounted to the upper housing.

The piston pump of the Versajet™ Hydrosurgery system also includes multiple parts requiring a complex assembly.

The current design reduces the number of parts and sub-assemblies, for example, several parts are integrated into one piece to avoid the assembly of the parts and maintain alignment of the parts.

A piston pump of a water jet debridement and wound bed preparation system includes an integrated, one-piece piston assembly including a fitting retainer, feed line fittings, and a support screen. Embodiments of this aspect may include two handle halves that are connectable to form an interior cavity that houses the components of the piston pump including the integrated, one-piece piston assembly.

The use of a single spike in the Versajet™ Hydrosurgery system may allow air to enter the tube and interlock the saline tubing if the saline bag is empty.

The two spike assembly of the current design enables air to be expelled from an open spike.

A water jet debridement and wound bed preparation system includes a piston pump, and two spikes with tubing extending from an inlet of the piston pump and configured to control air entering the system.

According to one aspect, a water jet hand piece for treating tissue includes a handle housing, a jet tube mounted to the housing, and a distal tip that receives liquid from the jet tube and defines a treatment window for treating tissue with a liquid jet. The distal tip is integral with the housing.

Embodiments of this aspect may include one or more of the following features.

The housing includes an upper housing and a lower housing and the distal tip is integral with the upper housing. The upper housing includes a distal housing and a proximal housing, and the distal tip and the distal housing are an integral, one-piece component. The water jet hand piece includes an evacuation tube received by the lower housing. The jet tube includes a 180 degree curved distal end. The water jet hand piece includes an orifice member.

According to another aspect, a water jet hand piece for treating tissue includes a handle including an upper housing and a lower housing, a jet tube mounted to the lower housing, and an evacuation tube mounted to the upper housing. The lower housing defines a treatment window. The jet tube has a straight distal end region. The treatment window is configured for treating tissue with a liquid jet delivered to the treatment window via the jet tube.

Embodiments of this aspect may include the lower housing defining a distal inner surface and the upper housing defining a distal inner surface. The distal inner surfaces are configured to guide the liquid jet from the treatment window to the evacuation tube.

According to another aspect, a water jet hand piece for treating tissue includes a distal tip defining an internal flow path and a treatment window, a distal tip cap, and an orifice member positioned between the distal tip and the cap. The hand piece is configured for liquid flow through the flow path and out the orifice member to the treatment window.

According to another aspect, a pump for a water jet debridement and wound bed preparation system includes an integrated, one-piece piston assembly including a fitting retainer, feed line fittings, and a support screen.

Embodiments of this aspect may include one or more of the following features: a handle, a piston, a piston cap, a single O-ring, and two valve balls. In an illustrated embodiment, the pump includes two handle halves that are connectable to form an interior cavity that houses the integrated, one-piece piston assembly. The connected handle halves form a piston cap.

According to another aspect, a water jet debridement and wound bed preparation system includes a piston pump, and two spikes with tubing extending from an inlet of the piston pump and configured to control air entering the system.

According to another aspect, a water jet hand piece for treating tissue includes a jet tube, an orifice member received in a distal end of the jet tube, and a spacer received in the distal end of the jet tube on top of the orifice member between the orifice member and a fluid outlet of the jet tube. In an illustrated embodiment, the spacer is welded to the jet tube.

### BRIEF DESCRIPTION OF THE DRAWINGS

FIGS. 1A-1C illustrates a prior art handle of a water jet debridement and wound bed preparation system.

FIG. 2 illustrates a prior art piston pump of a water jet debridement and wound bed preparation system.

FIG. 3 illustrates a prior art single spike connection to a saline bag in a water jet debridement and wound bed preparation system.

FIGS. 4A-41 include various views of a handle of a water jet debridement and wound bed preparation system.

FIG. 5 is a cross-section view of an alternative embodiment of a handle of a water jet debridement and wound bed preparation system.

FIGS. 6A-6D include various views of another alternative embodiment of a handle of a water jet debridement and wound bed preparation system.

FIGS. 7A-7F shows various embodiments of the industrial design of the hand piece.

FIGS. 8A-8D include various views of a piston pump of a water jet debridement and wound bed preparation system.

FIGS. 9A-9D include various view of an alternative embodiment of a piston pump of a water jet debridement and wound bed preparation system.

FIGS. 10 and 11 illustrate a dual-spike saline bag assembly of a water jet debridement and wound bed preparation system.

FIG. 12 illustrates a drip chamber of a water jet debridement and wound bed preparation system.

FIGS. 13A-13C illustrate a console of a water jet debridement and wound bed preparation system.

FIGS. 14A-16B illustrate various embodiments of orifice member assembly processes.

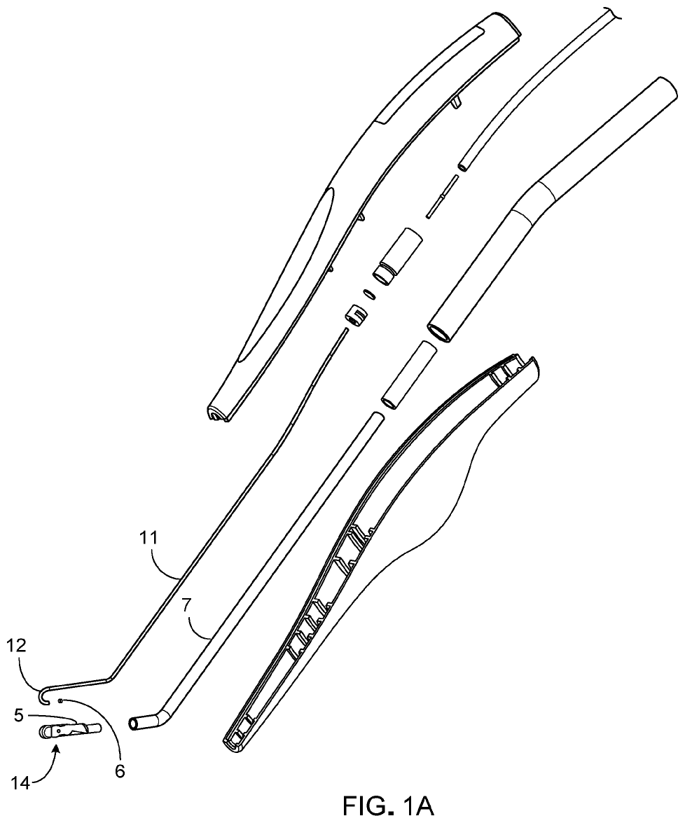

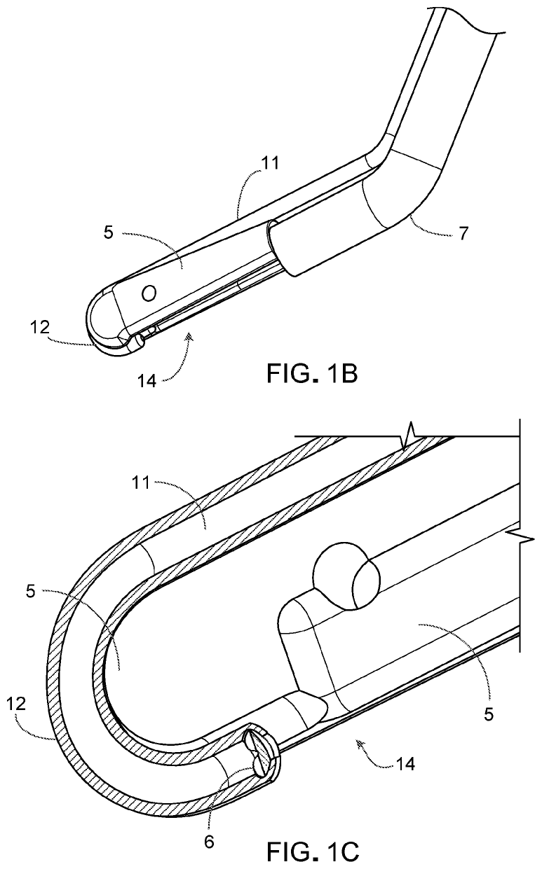

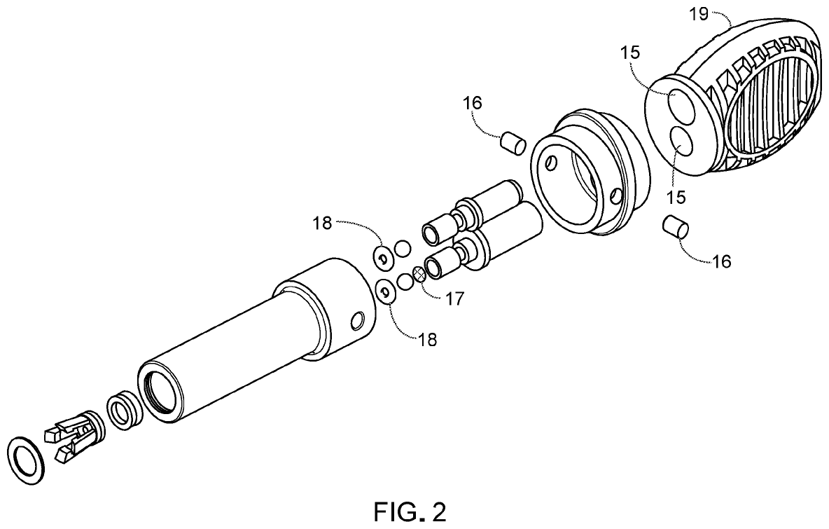

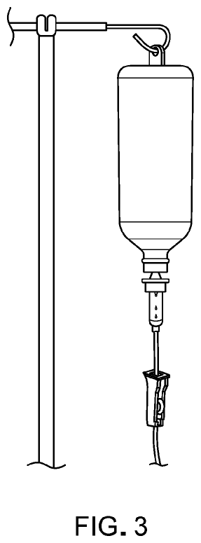

### DETAILED DESCRIPTION

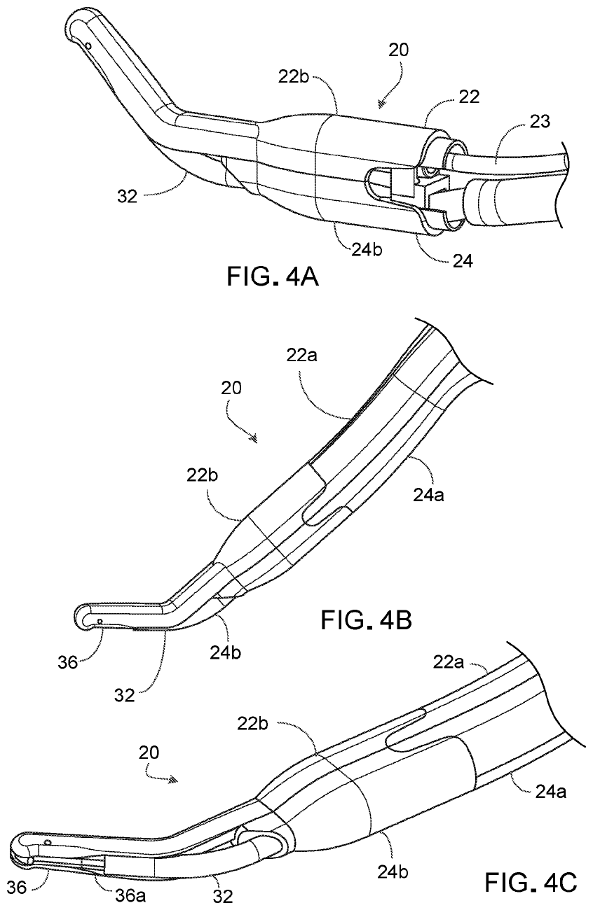

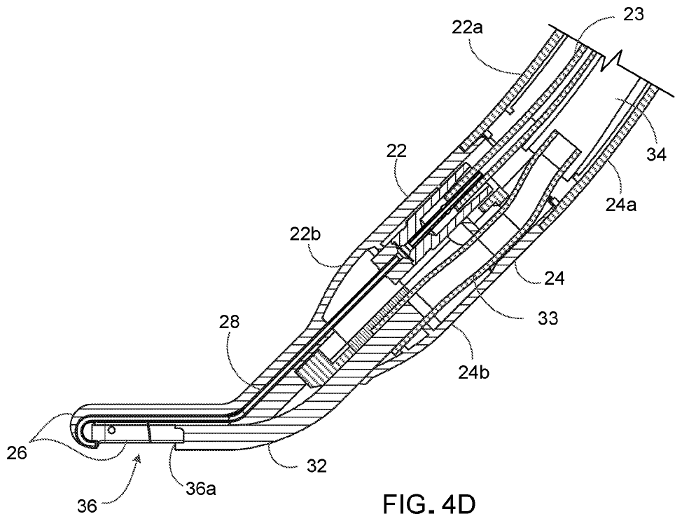

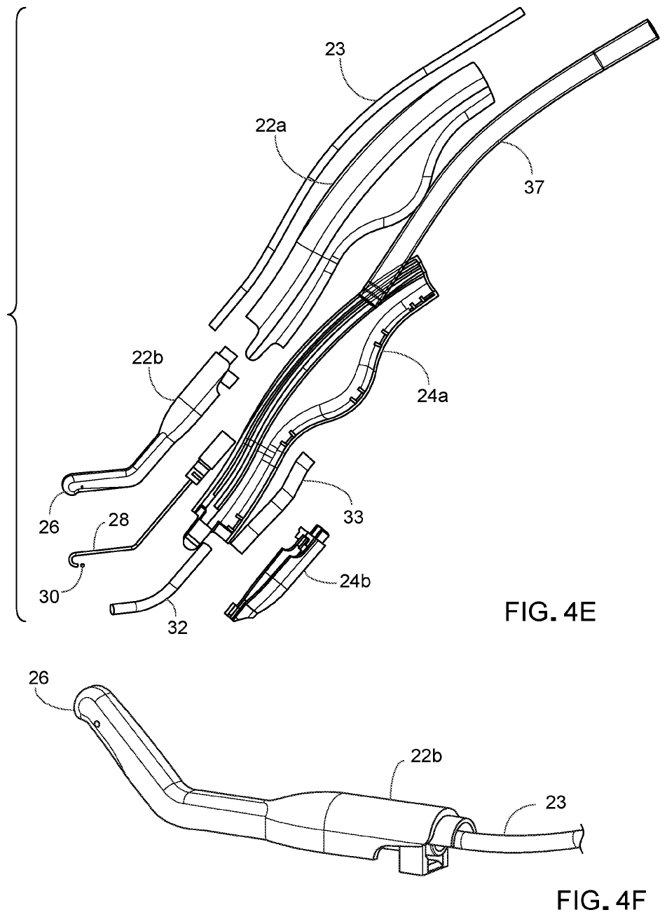

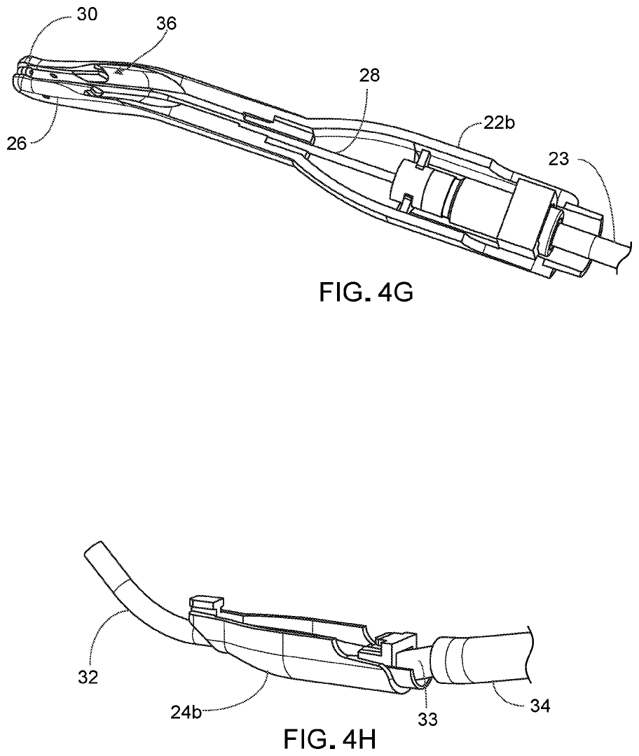

Referring to FIGS. 4A-4H, a hand piece 20 of a water jet debridement and wound bed preparation system includes a handle top housing 22 and a handle bottom housing 24. The handle top housing 22 has a proximal housing 22a and a distal housing 22b with a distal tip 26, which is an integral component of the distal housing 22b, for example, one plastic molded or machined part such that the distal tip 26 and distal housing 22b are an integral, one-piece component and assembly of the hand piece does not include attachment of the distal tip 26 to the distal housing 22b. Received within the distal housing 22b is a jet tube 28 having a liquid outlet orifice member 30. The distal tip 26 of the distal housing 22b defines a treatment window 36 for treating tissue with a jet of liquid. Received within the proximal housing 22a is a high pressure hose 23 through which the high pressure water is fed to the jet tube 28. Making the distal tip 26 integral with the distal housing 22b eliminates the need to weld the jet tube to the distal tip.

Referring also to FIGS. 4H and 41, the handle bottom housing 24 has a proximal housing 24a and a distal housing 24b. When assembled, the proximal housing 24a mates with the proximal housing 22a, and the distal housing 24b mates with the distal housing 22b. Leading from a proximal end 36a of the treatment window 36 to the distal housing 24b is an evacuation tube 32 partly covered by the distal housing 24b. A connection tube 33 leads from the evacuation tube 32 to a hose 34 through which the return flow exits the hand piece.

As compared to the prior art Versajet™ Hydrosurgery system, the high pressure jet tube 28 is shorter and the evacuation tube 32 is shorter.

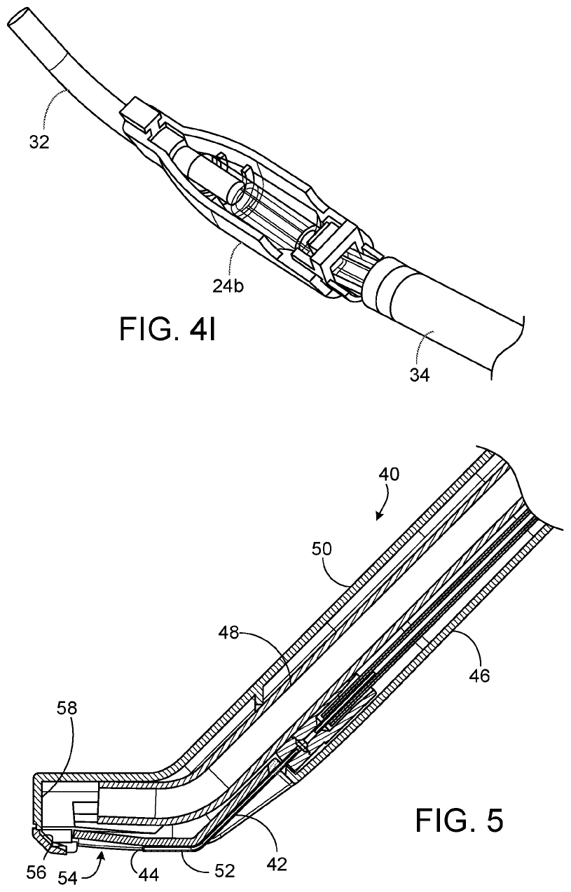

Referring to FIG. 5, in an alternative embodiment, a hand piece 40 of a water jet debridement and wound bed preparation system includes a jet tube 42 having a liquid outlet orifice member 44. The jet tube 42 is received in a handle bottom housing 46, and an evacuation tube 48 is received in a handle top housing 50. As compared to the jet tube 28 of FIG. 4D, the jet tube 42 has a straight distal end region 52. In addition, the hand piece 40 does not include a component corresponding to the distal tip 26 of FIG. 4D.

In use, the liquid is ejected from the orifice member 44 of the jet tube 42, travels across a tissue treatment window 54 where the jet acts to debride the wound bed, hits inner surfaces 56, 58 of the handle bottom and top housings 46, 50, respectively, and flows into the evacuation tube 48.

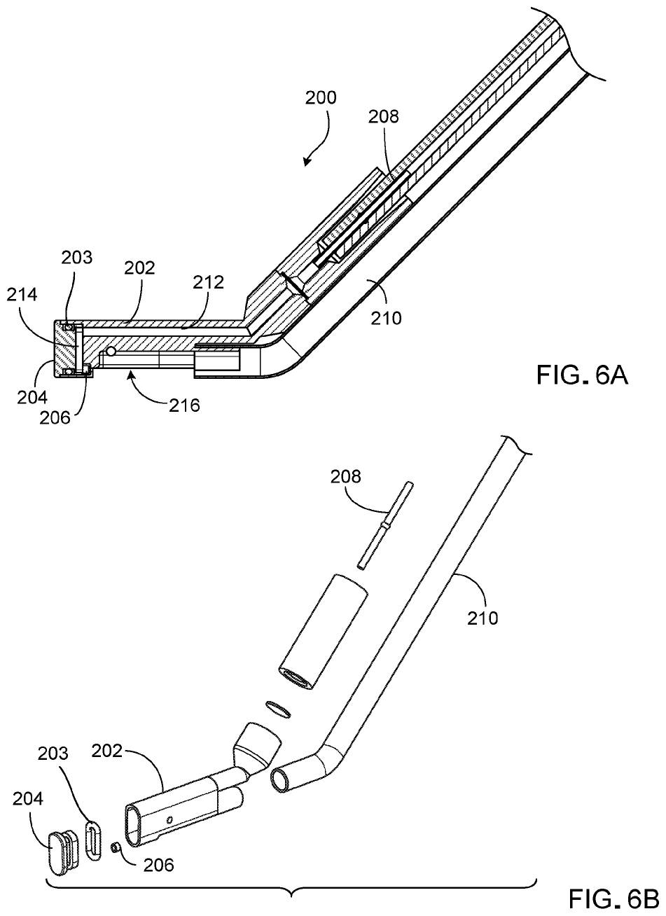

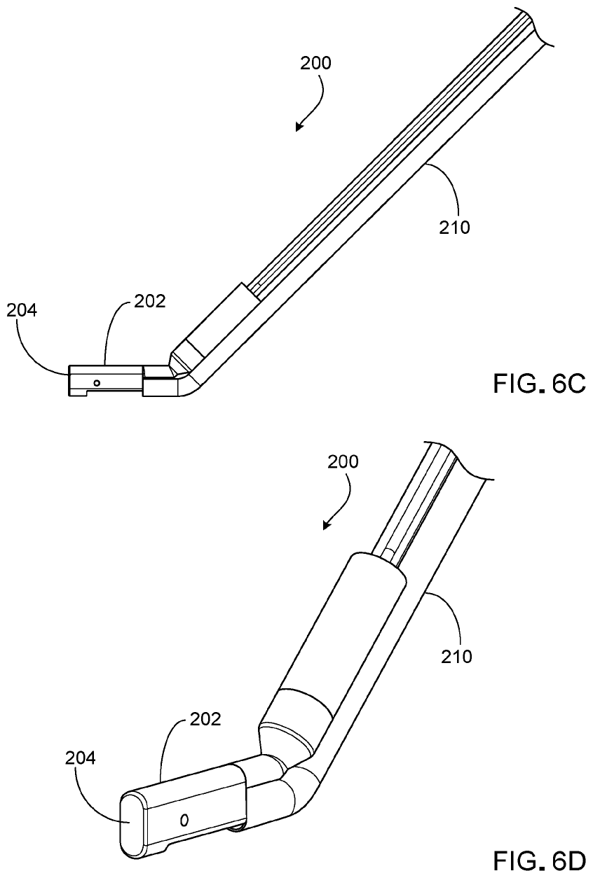

Referring to FIGS. 6A-6D, in an alternative embodiment, a hand piece 200 incudes a metal injection molded distal tip 202 and a metal injection molded distal tip cap 204. Trapped against the distal tip 202 by the tip cap 204 is a liquid outlet orifice member 206 and an O-ring 203. Liquid is delivered to the distal tip 202 by a high pressure line 208 and liquid exits via an evacuation tube 210. Liquid flows from the high pressure line 208 through paths 212, 214 in distal tip 202, out the orifice member 206, across a treatment window 216, and through evacuation tube 210.

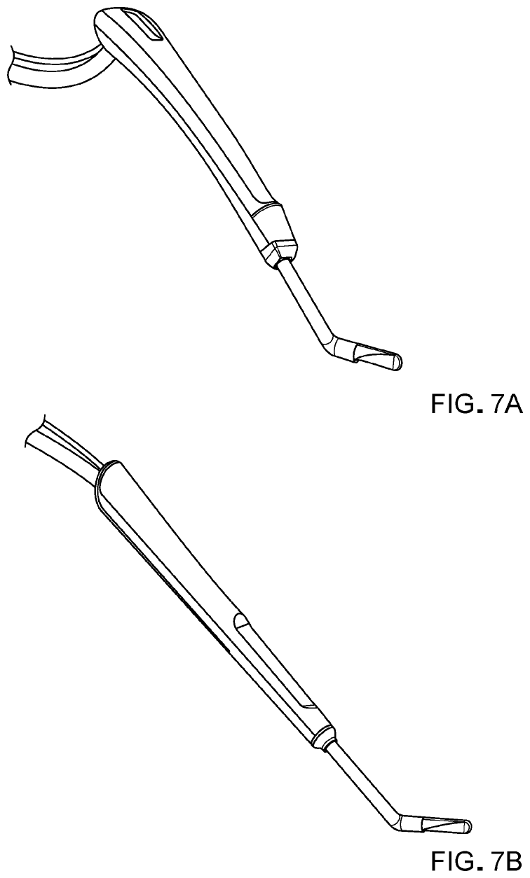

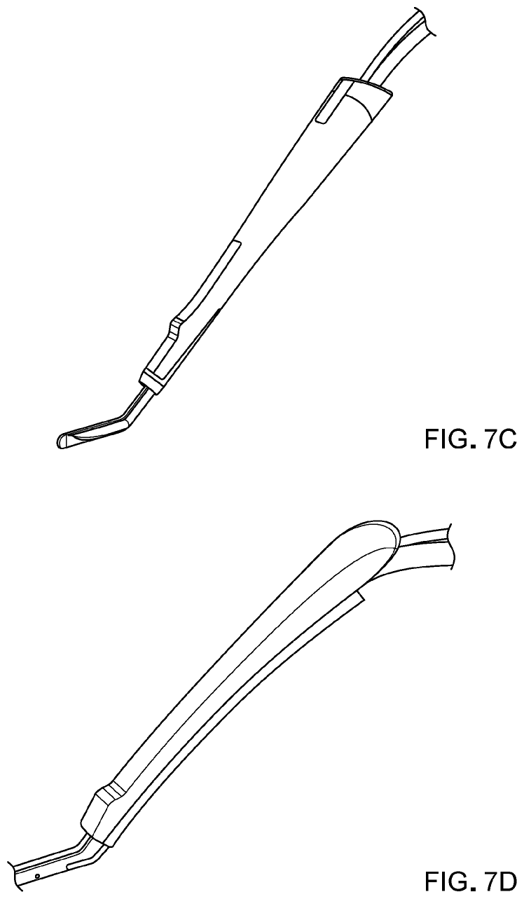

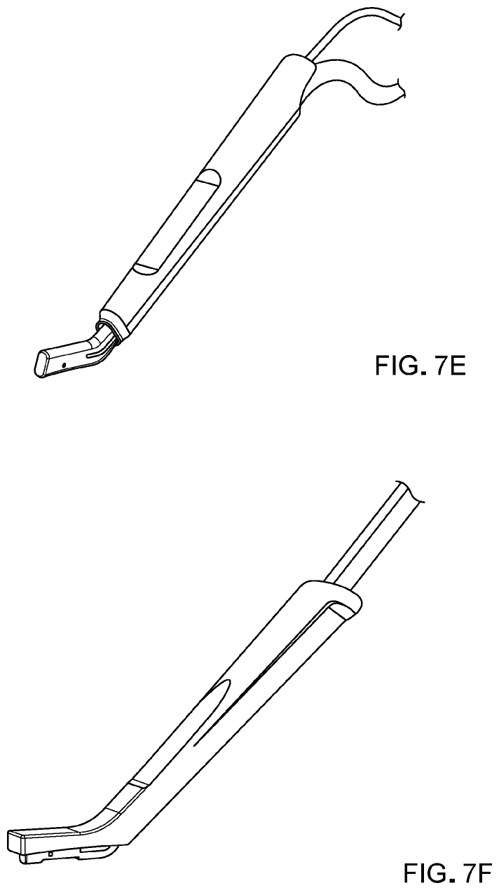

FIGS. 7A-7D illustrate alternative handle designs.

FIGS. 7E and 7F illustrate alternative handle designs corresponding to the embodiment of FIG. 6A.

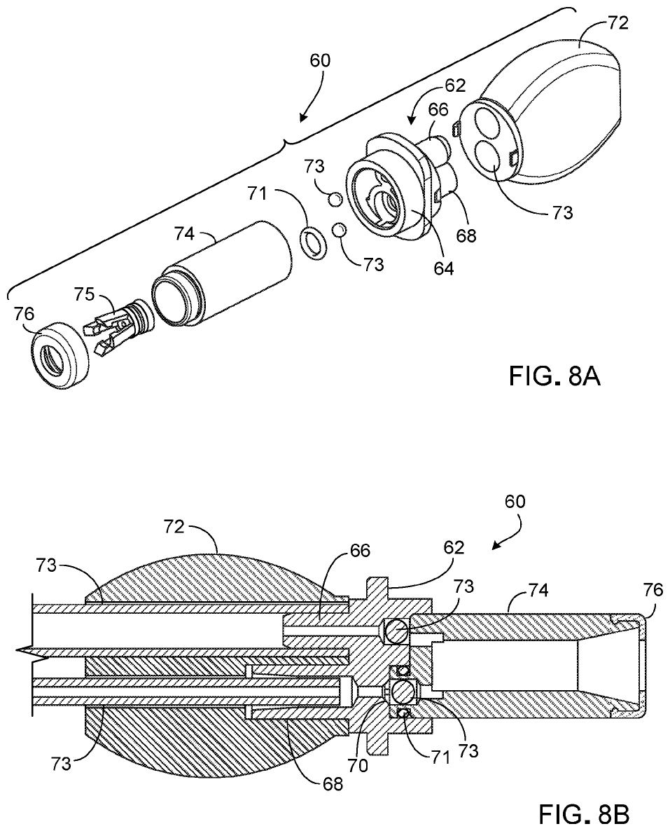

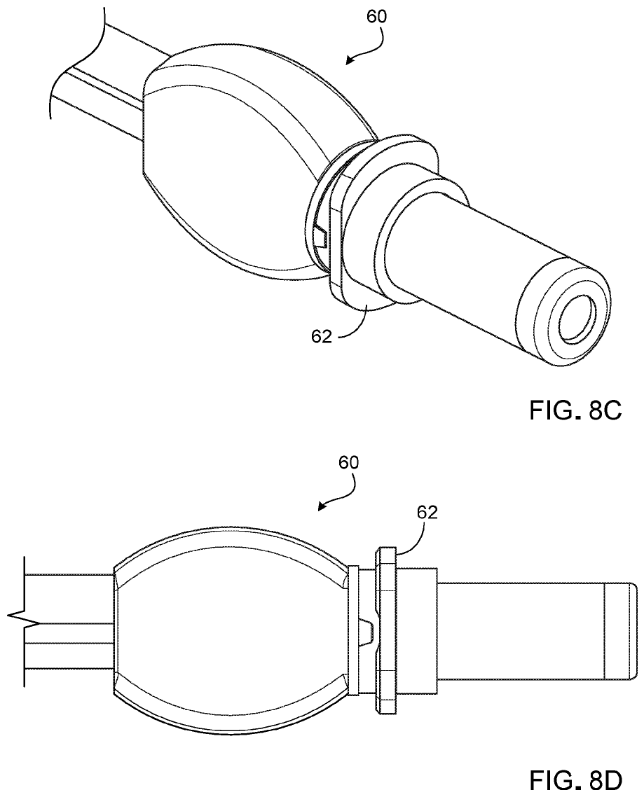

Referring to FIGS. 8A-8D, a piston pump 60 has a reduced number of parts and sub-assemblies as compared to the piston pump of FIG. 2. The piston pump 60 includes an integrated, one-piece piston assembly 62 including a fitting retainer 64, feed line fittings 66, 68, and a support screen 70, negating the need for assembling the feed line fittings to the fitting retainer with the dowel pins 16 of FIG. 2 and the need for placement of the support screen 17 in the assembly of FIG. 2. The integrated piston assembly 62 can be manufactured by, for example, metal injection molding, machining and/or ceramic injection molding.

Rather than the two O-rings 18 of FIG. 2, the piston pump 60 includes a single O-ring 71. The piston pump 60 includes a handle 72 defining two channels 73 that receive the fittings 66, 68. The piston pump 60 further includes two ball valves 73, a pump body 74, a piston 75, and a piston cap 76.

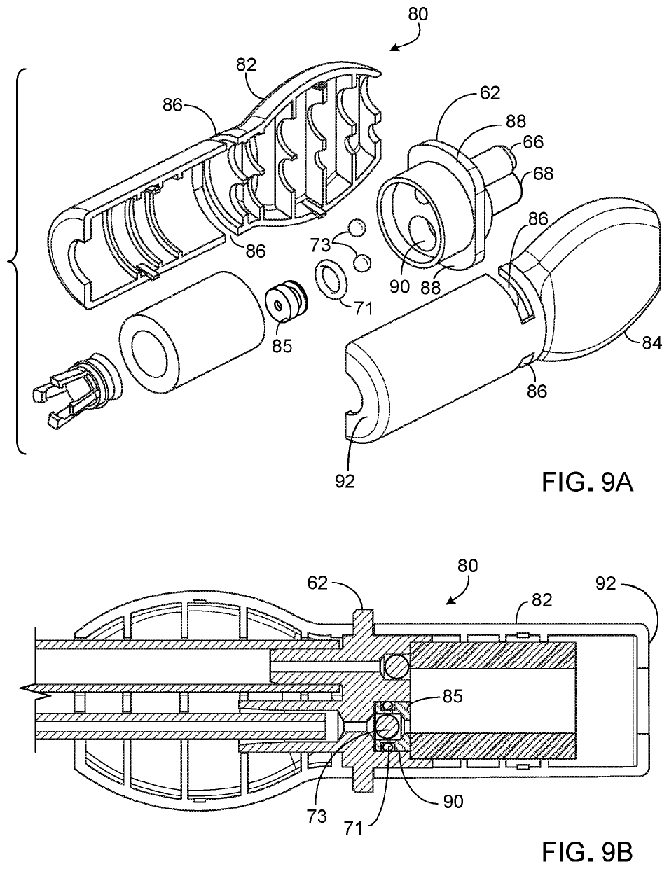

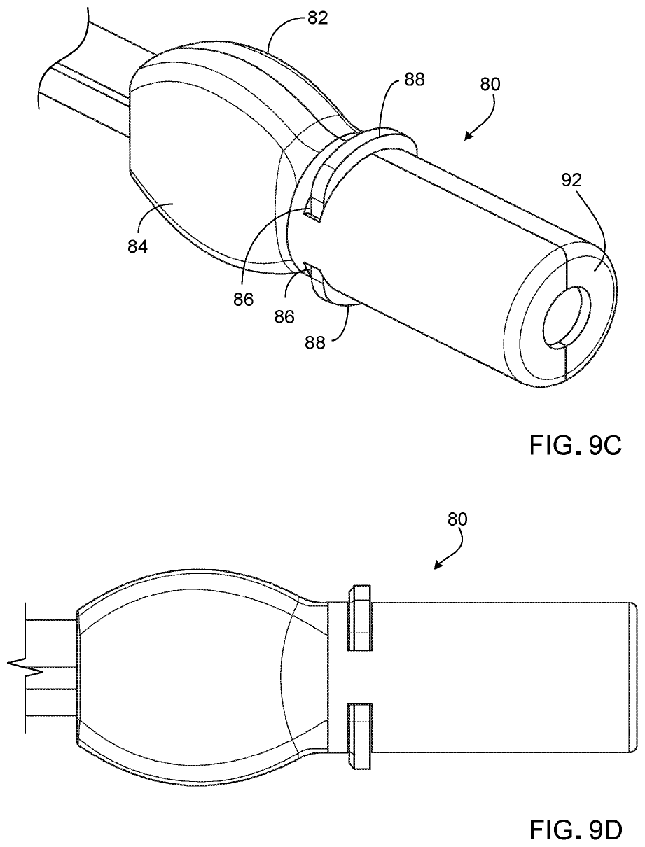

Referring to FIGS. 9A-9D, in another embodiment, a piston pump 80 includes two handle halves 82, 84 that are longer than the handle 19 of FIG. 2 and the handle 72 of FIG. 8A, and that, when connected, form an interior cavity that houses the components of the piston pump, including the integrated, one-piece piston assembly 62. The piston pump 80 includes a valve socket 85 that receives the O-ring 71 and one of the ball valves 73. The valve socket 85 is received within an opening 90 to the feed-line fitting 68. The handle halves 82, 84 define slots 86 that receive fins 88 of the piston assembly 62. When connected, the handle halves form a piston cap 92. The handle halves 82, 84 facilitate assembly of the piston pump 80, and do not require the fluid channels 15 and 73 in the handles of FIG. 2 and FIG. 8A.

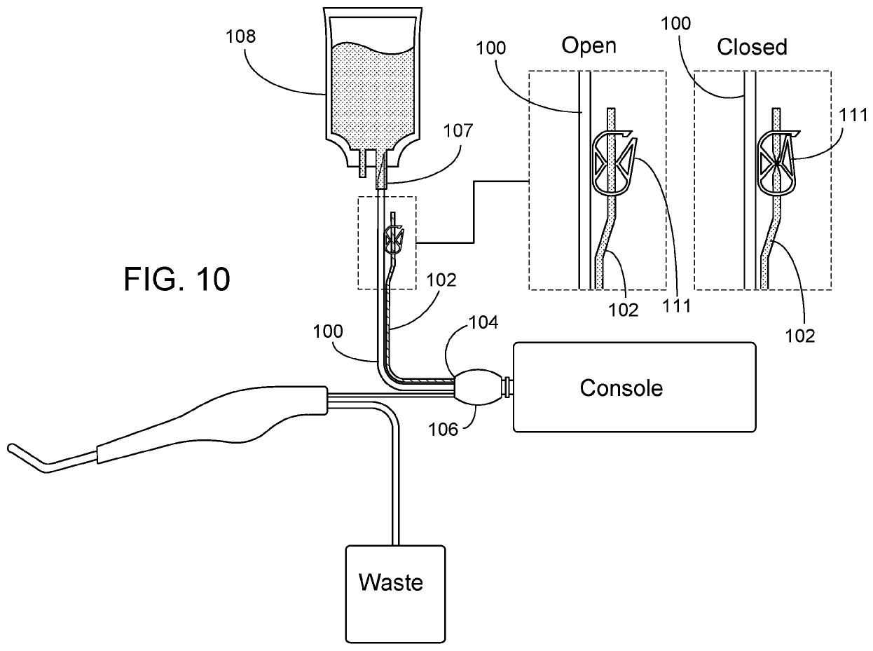

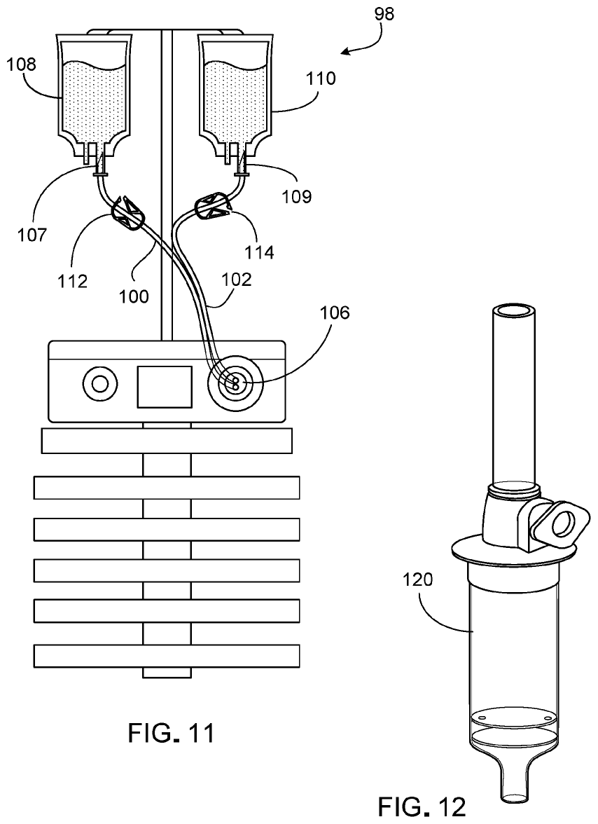

Referring to FIGS. 10 and 11, a water jet debridement and wound bed preparation system 98 includes two saline tubes 100, 102 extending from an inlet 104 of a piston pump handle 106. Saline tube 100 terminates in a spike 107 that connects to a saline bag 108. Saline tube 102 can be open to atmosphere (FIG. 10) or include a spike 109 that connects to a second saline bag 110 (FIG. 11). In the assembly of FIG. 10, air is expelled from the open tube 102 with a clamp 111 in an open position when there is an airlock in the system. Tube 102 of FIG. 10 is illustrated without a spike, but may optionally include a spike. In the assembly of FIG. 11, the two spikes 107, 109 are connected to the separate saline bags 108, 110 with respective clamps 112, 114 on the tubing 100, 102. In use, one of the clamps, for example, clamp 112, is closed and the other clamp, for example, clamp 114, is open. The saline water in the clamp open bag 110 is used first. When the saline bag 110 is empty, air will enter the tube 102 and the pump, which can result in airlocking of the system. To re-prime the pump and remove the airlock, clamp 112 is opened allowing saline to flow down the tube 100 from the bag 108, through the pump chamber and back up the tube 102 towards the empty bag 110, thus flushing out the air locked in the system. Once no more air can be seen exiting the pump, the clamp 114 is closed and the pump is now primed and will draw fluid from the bag 108.

Referring to FIG. 12, in an alternative embodiment, a drip chamber 120 is used in the saline lines, which allow air to rise out from the liquid so that it is not passed downstream. The drip chamber should be maintained about half full to prevent air from getting into the saline tubing, which could block the tube and stop the procedure. As compared to the embodiments of FIGS. 10 and 11, if air is already in the tubing, the priming will stop. The embodiments of FIGS. 10 and 11 allow the air to rise out even if the air is already in the saline tubing such that the procedure is not impacted.

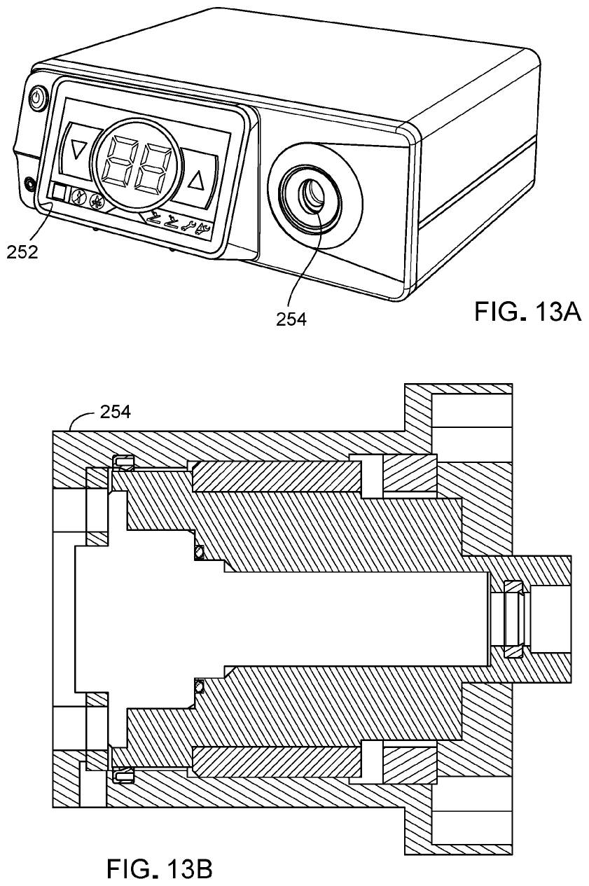

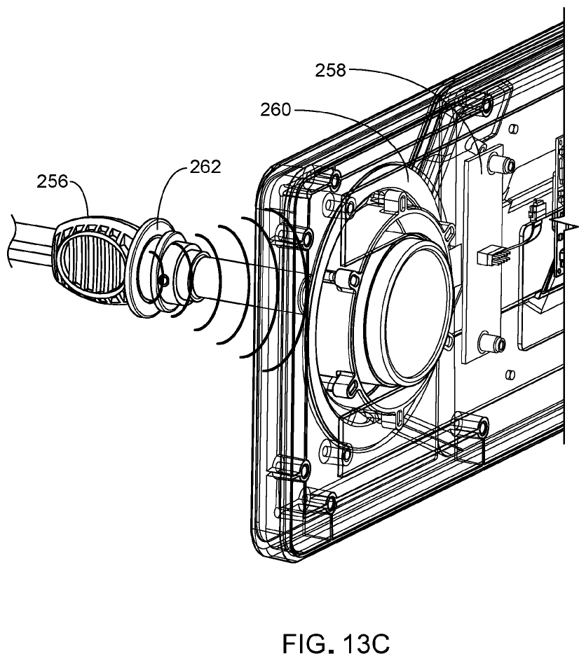

Referring to FIGS. 13A-13C, a console 250 of a water jet debridement and wound bed preparation system includes an LCD screen 252 for displaying device state information, for example, power level, procedure running time, out-patient/operating room mode, and service reminders. The console includes an interface 254 for receiving the piston pump 256, and an RFID reader 258 and antenna 260 for identifying an RFID tag 262 installed in the piston pump hand piece.

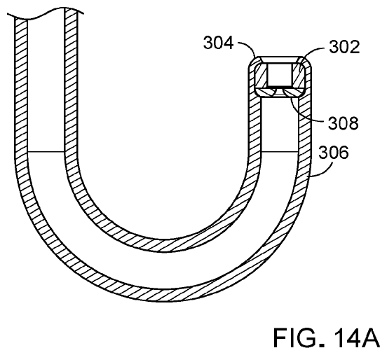

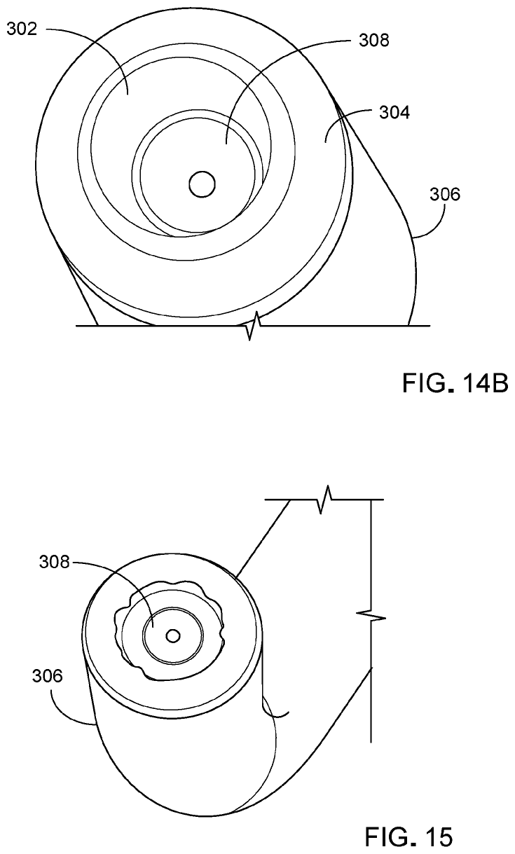

FIGS. 14A-16B illustrate various embodiments of orifice member assembly processes.

Referring to FIGS. 14A and 14B, to provide additional securement of the orifice member 308 to the jet tube 306, a metal spacer 302, for example, a gasket or bushing, is added to the assembly. In the assembly process, the end 304 of the jet tube 306 is flared and the orifice member 308 is placed within the end of the jet tube. The metal gasket or metal bushing 302 is then placed on top of the orifice member 308 between the orifice member 308 and the fluid outlet at the end 304 of the jet tube. The end 304 of the jet tube 306 is crimped to press the gasket or bushing 302 against the orifice member 308 and crimp the gasket or bushing together with the orifice member to the jet tube. The addition of the gasket or bushing ensures the orifice member has less space to move during the assembly process, which increase the crimping process consistency and improves on yield rate.

Referring to FIG. 15, the orifice member 308 can be laser-welded directly into the end of jet tube 306. The orifice member is placed in the flared end of the jet tube followed by laser welding. The weld bump secures the orifice member in place.

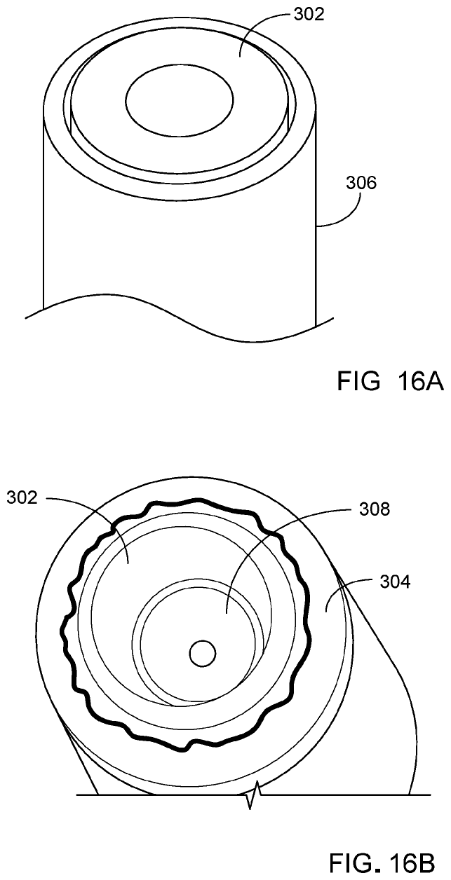

Referring to FIGS. 16A and 16B, the gasket or bushing 302 position above the orifice member 308 of FIGS. 14A and 14B can be combined with laser-welding. The orifice member 308 is placed in the end of the flared jet tube 306 followed by placement of the metal gasket or bushing on top of the orifice member. Laser welding is performed all around the top of the metal gasket or bushing. The metal gasket or bushing ensures that the orifice member has less space to move during assembly process. Laser welding on the metal gasket has the advantage of avoiding direct heat from the laser welding to the orifice member which may change the material property or weaken the orifice member. Laser welding has high consistency and repeatability, which ensure higher yield rate during manufacturing.

In an alternative embodiment, the orifice can be directly fabricated on the jet tube by micro-machining or EDM technology.

Other embodiments are within the scope of the following claims.

1. A water jet hand piece for treating tissue, comprising:
a handle including a housing;
a jet tube mounted to the housing; and
a distal tip that receives liquid from the jet tube and defines a treatment window for treating tissue with a liquid jet, the distal tip being integral with the housing.

2. The water jet hand piece of claim 1 wherein the housing includes an upper housing and a lower housing, the distal tip being integral with the upper housing.

3. The water jet hand piece of claim 2 wherein the upper housing comprising a distal housing and a proximal housing, the distal tip and the distal housing being an integral, one-piece component.

4. The water jet hand piece of claim 2 further comprising an evacuation tube received by the lower housing.

5. The water jet hand piece of claim 1 wherein the jet tube includes a 180 degree curved distal end.

6. The water jet hand piece of claim 1 further comprising an orifice member.

7. A water jet hand piece for treating tissue, comprising:
a handle including an upper housing and a lower housing, the lower housing defining a treatment window;
a jet tube mounted to the lower housing, the jet tube having a straight distal end region, the treatment window configured for treating tissue with a liquid jet delivered to the treatment window via the jet tube; and
an evacuation tube mounted to the upper housing.

8. The water jet hand piece of claim 7 wherein the lower housing defines a distal inner surface and the upper housing defines a distal inner surface, the distal inner surfaces being configured to guide the liquid jet from the treatment window to the evacuation tube.

9. A water jet hand piece for treating tissue, comprising:
a distal tip defining an internal flow path and a treatment window;
a distal tip cap; and
an orifice member positioned between the distal tip and the cap;
wherein the hand piece is configured for liquid flow through the flow path and out the orifice member to the treatment window.

10. A pump for a water jet debridement and wound bed preparation system, comprising:
an integrated, one-piece piston assembly including a fitting retainer, feed line fittings, and a support screen.

11. The pump of claim 10 further comprising a handle.

12. The pump of claim 10 further comprising a piston.

13. The pump of claim 12 further comprising a piston cap.

14. The pump of claim 10 consisting of a single O-ring.

15. The pump of claim 10 further comprising two valve balls.

16. The pump of claim 10 further comprising two handle halves that are connectable to form an interior cavity that houses the integrated, one-piece piston assembly.

17. The pump of claim 16 wherein the connected handle halves form a piston cap.

18. A water jet debridement and wound bed preparation system, comprising:
piston pump; and
two spikes with tubing extending from an inlet of the piston pump and configured to control air entering the system.

19. A water jet hand piece for treating tissue, comprising:
a jet tube;
an orifice member received in a distal end of the jet tube; and
and a spacer received in the distal end of the jet tube on top of the orifice member between the orifice member and a fluid outlet of the jet tube.

20. The water jet hand piece of claim 19 wherein the spacer is welded to the jet tube.
